(19) United States
(12) Patent Application Publication
(10) Pub. No.: US 2024/0324864 A1
(43) Pub. Date: Oct. 3, 2024

(54) MAGNETICALLY PROPELLED CAPSULE AND METHODS OF MAKING AND USING THE SAME

(71) Applicant: Liming Wang, Fuquay-Varina, NC (US)

(72) Inventor: Liming Wang, Fuquay-Varina, NC (US)

(21) Appl. No.: 18/437,452

(22) Filed: Feb. 9, 2024

Related U.S. Application Data
(60) Provisional application No. 63/455,187, filed on Mar. 28, 2023.

(51) Int. Cl. A61B 1/04; A61B 1/00; A61B 1/05; A61B 1/06; A61B 1/31; A61B 5/00

(52) U.S. Cl. CPC A61B 1/041; A61B 1/00016; A61B 1/00018; A61B 1/00045; A61B 1/00154; A61B 1/00158; A61B 1/05; A61B 1/0684; A61B 1/31; A61B 5/4255; A61B 2562/0219; A61B 2562/162

(57) ABSTRACT

The invention is an improved colonoscopy system and method. The disclosed system includes the use of a tiny, wired capsule that can be magnetically controlled through the upper and/or lower GI tract of a patient. The capsule includes a camera assembly and a light assembly to allow a medical professional to visualize an interior cavity of the patient. The capsule further includes one or more internal magnets. The capsule is configured with an outer covering that protects the internal components from damage during use. An external magnet is also provided to drive and orient the capsule through the patient's body via magnetic attraction with the internal magnet.

### CROSS-REFERENCE TO RELATED APPLICATIONS

This application claims priority to U.S. Provisional Patent Application No. 63/455,187, filed Mar. 28, 2023, the entire content of which is hereby incorporated by reference.

### FIELD OF THE INVENTION

The presently disclosed subject matter is directed to a magnetically propelled capsule. The presently disclosed subject matter further includes methods of making and using the disclosed magnetically propelled capsule.

### BACKGROUND OF THE INVENTION

Excluding skin cancers, colorectal cancer is the third most common cancer diagnosed in both men and women in the United States. The American Cancer Society's estimates for the number of colorectal cancer cases in the United States for 2019 were 101,420 new cases of colon cancer and 44,180 new cases of rectal cancer. Overall, the lifetime risk of developing colorectal cancer is about 1 in 22 (4.49%) for men and 1 in 24 (4.15%) for women. However, it is noted that the death rate (the number of deaths per 100,000 people per year) associated with colorectal cancer has steadily dropped over the past several decades. The decrease in death rate has been attributed to an increase in screening methods, leading to the discovery and removal of colorectal polyps before they can develop into cancers. Despite the advances in detection methods, about 1 in 3 people in the United States that should get tested for colorectal cancer have never been screened due to fear, ignorance, cost, and issues with health insurance coverage.

There are many colorectal screening tests currently being used. For example, stool-based tests check the stool (feces) of a patient for signs of cancer. While less invasive and easier to perform, stool-based tests are less sensitive to detecting cancer and must be done more often. In addition, there are several types of visual tests that can be performed to detect colon cancer.

A colonoscopy is a type of visual test performed to examine the entire length of the colon and rectum. A colonoscopy typically includes inserting a colonoscope into the rectum of a patient. The colonoscope is then steered by adjusting and manually pushing the device to a desired location, after which it is withdrawn. However, conventional colonoscopes suffer from many disadvantages. For example, insertion of a scope into a hollow organ is a risky maneuver that can be associated with trauma, bleeding, and perforations. In addition, current colonoscopes have a narrow field of vision, leading to a large number of missed pathologic findings. Further, colonoscopy procedures are conventionally performed under patient sedation, which carries risks and requires the patient to take a day off from normal activities. Due to the sedation, conventional colonoscopy procedures are associated with significant patient fear that is often used as a justification for delaying or avoiding endoscopic diagnosis.

CT colonography (virtual colonoscopy) is an advanced type of visual test where a computed tomography (CT) scan of the colon and rectum is performed. A CT scan uses x-rays and takes a series of pictures as it rotates around the patient. A computer then combines the pictures into detailed images of the body part being studied. For example, both two-dimensional x-ray pictures and a three-dimensional view of the inside of the colon and rectum can be produced to screen for polyps or cancer. The test can be done fairly quickly, and sedation is not needed. However, after CT colonoscopy procedures, patients often feel bloated and/or have cramps due to the presence of the air in the colon and rectum. There is also a risk that the colon can be injured or punctured as a result of the inflation. Further, the test also exposes the patient to a small amount of radiation.

Flexible sigmoidoscopy is a type of visual test where a sigmoidoscope (a flexible, lighted tube with a small video camera on the end) is used to visualize the colon and rectum. The scope is inserted through the anus, into the rectum, and moved into the lower part of the colon. Images from the scope are visualized by the medical professional on a video screen. Using the sigmoidoscope, abnormalities can be detected and possibly removed. The sigmoidoscope is only about 60 centimeters (about 2 feet) long, so the medical professional can visualize the entire rectum but less than half of the colon. In addition, flexible sigmoidoscopy procedures may be uncomfortable due to the addition of air to the colon and rectum. Bleeding and puncture of the colon and/or rectum are further possible complications.

It would therefore be beneficial to provide an improved system and method that can be used to visualize the interior of a subject's body. It would further be beneficial if the disclosed system and method overcomes the disadvantages of prior art colonoscopy testing methods.

### SUMMARY OF THE INVENTION

The presently disclosed subject matter is directed to a magnetically driven capsule. The capsule comprises a camera assembly configured to take images, provide video, or both. The capsule further includes a light assembly configured to illuminate areas surrounding the camera assembly. The capsule comprises an internal magnet and an outer covering surrounding the camera assembly, light assembly, and magnet.

In some embodiments, the camera assembly comprises a digital camera or a digital video camera.

In some embodiments, the camera assembly is configured to provide a two-dimensional or three-dimensional image.

In some embodiments, the light assembly comprises one or more light emitting diodes (LED).

In some embodiments, the internal magnet is a permanent magnet that remains magnetized.

In some embodiments, the internal magnet comprises steel, neodymium, samarium cobalt, ceramic, aluminum nickel cobalt, iron, nickel, cobalt, alloy of rare earth metals, lodestone, or combinations thereof.

In some embodiments, the magnetically driven capsule further includes a power source comprising a radiofrequency (RF) generating component.

In some embodiments, the magnetically driven capsule further includes an accelerometer, a gyroscope, or both.

In some embodiments, the capsule comprises a length of about 2 centimeters or less and a diameter of about 1 cm or less.

In some embodiments, the presently disclosed subject matter is directed to a system for guiding a magnetically driven capsule. Particularly, the system comprises a magnetically driven capsule defined by a camera assembly configured to take images, provide video, or both; a light assembly configured to illuminate areas surrounding the camera assembly; an internal magnet; and an outer covering surrounding the camera assembly, light assembly, and magnet. The system further includes an external magnet that exhibits magnetic attraction to the internal magnet sufficient to guide a location of the internal magnetically driven capsule.

In some embodiments, the external magnet is an electromagnet.

In some embodiments, external magnet comprises neodymium, samarium cobalt, ceramic, aluminum nickel cobalt, or combinations thereof.

In some embodiments, the presently disclosed subject matter is directed to a method of visualizing a body cavity. Particularly, the method comprises positioning a magnetically driven capsule within a body cavity to be visualized. The magnetically driven capsule is defined by: a camera assembly configured to take images, provide video, or both; a light assembly configured to illuminate areas surrounding the camera assembly; an internal magnet; and an outer covering surrounding the camera assembly, light assembly, and magnet. The method includes maneuvering the magnetically driven capsule along a desired path using an external magnet positioned on an external surface of the patient's body, whereby magnetic attraction between the internal and external magnets guide the path of the capsule and whereby the body cavity is visualized.

In some embodiments, the body cavity is selected from the rectum, large intestine, or combinations thereof. The “rectum” refers to the final straight portion of the large intestine in humans. The “large intestine” refers to the last portion of the gastrointestinal tract and of the digestive system.

In some embodiments, the magnetically driven capsule can be maneuvered via the external magnet in a forward direction (e.g., from the rectum towards the large intestine of a patient), a backward direction (e.g., from the large intestine towards the rectum of a patient), to the right and/or to the left (move in a side-to-side manner), rotate (e.g., curved movement about a center point), or combinations thereof.

In some embodiments, the positioning is accomplished by a plastic tubing sized to allow the magnetically driven capsule to pass therethrough.

In some embodiments, the magnetic force of the external magnet is applied to the internal magnet at an angle of about 15-80 degrees.

In some embodiments, the magnetic force of the external magnet is applied to the internal magnet at an angle of about 30-60 degrees.

### BRIEF DESCRIPTION OF THE DRAWINGS

FIG. 1a is a schematic of conventional colonoscopy systems.

FIG. 1b is an illustration of a colonoscopy used in conventional colonoscopy procedures.

FIG. 2 is a front plan view of a magnetically driven capsule in accordance with some embodiments of the presently disclosed subject matter.

FIG. 3a is a side plan view of an external magnet interacting with a magnetically driven capsule in accordance with some embodiments of the presently disclosed subject matter.

FIG. 3b is a front plan view of an external magnet in accordance with some embodiments of the presently disclosed subject matter.

FIGS. 4a-4d are cross-sectional views of capsules in accordance with some embodiments of the presently disclosed subject matter.

FIG. 5a is a front plan view of a magnetically driven capsule in accordance with some embodiments of the presently disclosed subject matter.

FIG. 5b is a top plan view of a magnetically driven capsule in accordance with some embodiments of the presently disclosed subject matter.

FIG. 6 is a schematic of one method of using a magnetically driven capsule in a colonoscopy procedure in accordance with some embodiments of the presently disclosed subject matter.

FIG. 7 is a schematic illustrating the rectum, colon, large intestine, and small intestine in some embodiments.

### DETAILED DESCRIPTION OF THE INVENTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e., at least one) of the grammatical object of the article. By way of example, “an element” means at least one element and can include more than one element. It will be further understood that the terms “comprises,” “comprising,” “includes,” and/or “including” when used herein specify the presence of stated features, integers, steps, operations, elements, and/or components, but do not preclude the presence or addition of one or more other features, integers, steps, operations, elements, components, and/or groups thereof.

Unless otherwise indicated, all numbers expressing quantities of components, conditions, and so forth used in the specification and claims are to be understood as being modified in all instances by the term “about.” Accordingly, unless indicated to the contrary, the numerical parameters set forth in the instant specification and attached claims are approximations that can vary depending upon the desired properties sought to be obtained by the presently disclosed subject matter.

As used herein, the term “about”, when referring to a value or to an amount of mass, weight, time, volume, concentration, and/or percentage can encompass variations of, in some embodiments +/−20%, in some embodiments +/−10%, in some embodiments +/−5%, in some embodiments +/−1%, in some embodiments +/−0.5%, and in some embodiments +/−0.1%, from the specified amount, as such variations are appropriate in the disclosed packages and methods. Thus, the term “about” is used to provide flexibility to a numerical range endpoint by providing that a given value may be “slightly above” or “slightly below” the endpoint without affecting the desired result.

As used herein, the term “and/or” includes any and all combinations of one or more of the associated listed items.

Relative terms such as “below” or “above” or “upper” or “lower” or “horizontal” or “vertical” may be used herein to describe a relationship of one element, layer, or region to another element, layer, or region as illustrated in the drawing figures. It will be understood that these terms and those discussed above are intended to encompass different orientations of the device in addition to the orientation depicted in the drawing figures.

The embodiments set forth below represent the necessary information to enable those skilled in the art to practice the embodiments and illustrate the best mode of practicing the embodiments. Upon reading the following description in light of the accompanying drawing figures, those skilled in the art will understand the concepts of the disclosure and will recognize applications of these concepts not particularly addressed herein. It should be understood that these concepts and applications fall within the scope of the disclosure and the accompanying claims.

All methods described herein can be performed in any suitable order unless otherwise indicated herein or otherwise clearly contradicted by context. The use of any and all examples, or exemplary language (e.g., “such as”) provided herein, is intended merely to better illuminate the invention, and does not pose a limitation on the scope of the invention unless otherwise claimed. No language in the specification should be construed as indicating any unclaimed element as essential to the practice of the invention.

Unless otherwise defined, all technical terms used herein have the same meaning as commonly understood by one of ordinary skill in the art to which this disclosure belongs.

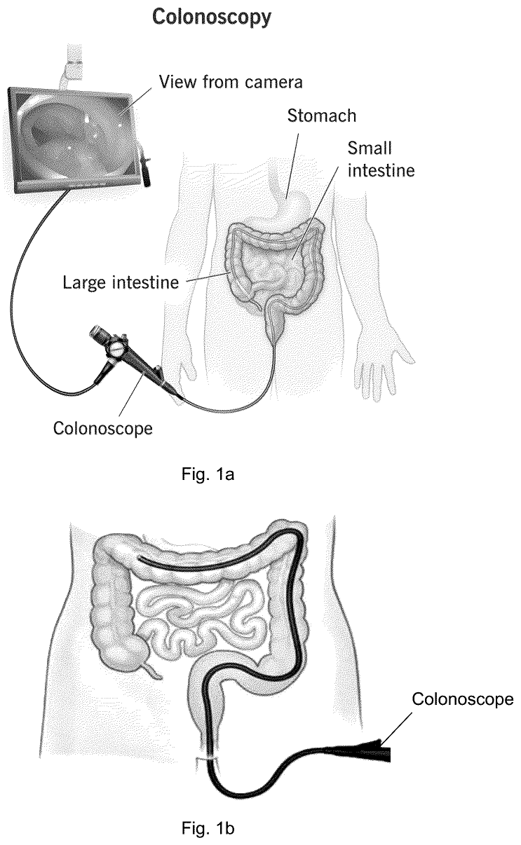

FIGS. 1a and 1b illustrate one embodiment of a typical colonoscopy procedure. As shown, a colonoscope is inserted into the anus, passes through the rectum, and into the large and small intestines for visualization. In addition, special instruments can be passed through the colonoscope to biopsy or remove any areas of concern (e.g., polyps). However, as discussed above, current colonoscopy methods suffer from many drawbacks. Due to the drawbacks and risk, patients may skip the colonoscopy, thereby decreasing their chances of catching abnormalities at an early stage.

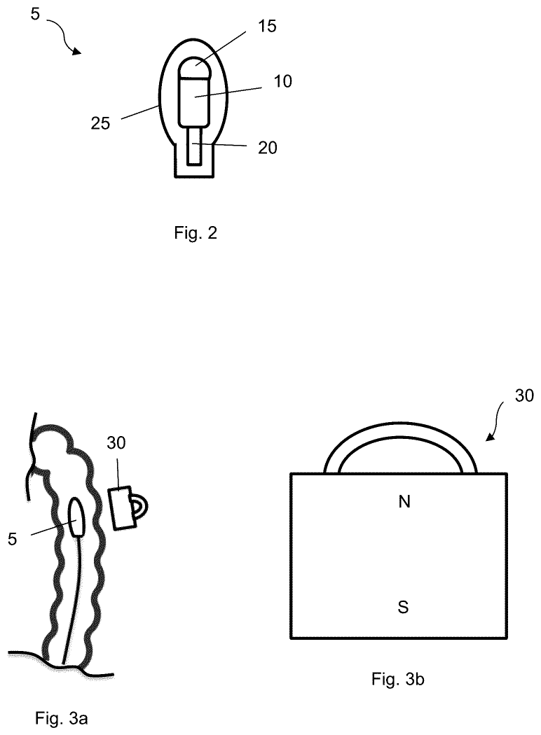

The disclosed invention is directed to an improved colonoscopy screening system and method. The term “colonoscopy” refers to an endoscopic examination wherein a scope is moved through the regions of the colon and a medical professional examines a visual image of the lining of the colon. The disclosed system includes the use of a wired capsule 5 that can be magnetically controlled through the upper and/or lower GI tract of a patient using an externally positioned magnet. The term “capsule” refers to a substantially or fully enclosed container, such as (but not limited to) a wired probe. As illustrated in FIG. 2, capsule 5 includes camera assembly 10 and light assembly 15 that allow a medical professional to visualize the interior of the patient. The capsule includes associated wiring that can be used to power the camera assembly and/or light assembly, as well as provide a connection to the healthcare professional's computer screen for imaging. In some embodiments, the capsule can wirelessly transmit information to the healthcare professional.

The capsule further includes one or more internal magnets 20. The capsule is configured with outer covering 25 that covers the entire exterior of the capsule, protecting the internal components from damage during use. An external magnet 21 is also provided to drive and orient the capsule through the patient's body, as discussed in detail below. Advantageously, the magnetically propelled capsule allows for a fast, comprehensive medical examination of the patient. For example, a colonoscopy can be performed without sedation, with minimal risk, and little to no patient discomfort.

As noted above, capsule 5 includes camera assembly 10 and light assembly 15 that allow an interior body cavity (e.g., the rectum and colon) to be visualized. The term “camera assembly” broadly refers to any imaging device used for capturing light. Thus, in some embodiments, camera assembly 10 can include at least one optical lens assembly. Existing colonoscopes generally include only one camera configured as a front-facing camera. In some cases, particularly in colonoscopy, a polyp, a lesion, or a tumor may be located on an inner side of a fold of the colon, such that it is hidden from the field of view of the front camera. Accordingly, camera assembly 10 can include a plurality of cameras or lenses in some embodiments. For example, the disclosed camera assembly can include at least one camera pointing at a different direction than a front pointing camera and is therefore adapted to solve the problem of limited field of view. Thus, camera assembly 10 can include a front facing camera, one or more side facing cameras, and or one or more rear-facing cameras.

Suitable cameras can include (but are not limited to) digital video cameras (video cameras that capture videos in digital memory, such as moving visual images in the form of encoded digital data), digital still cameras (cameras that capture photographs in digital memory), or other cameras capable of providing digital image data or video image data. In some embodiments, camera 10 can include a wide angle camera that allows a field of view with a large angle (e.g., at least about 90, 100, 110, 120, 130, 140, 150, 160, 170, or 180 degrees) to its optical axis.

Capsule 5 further includes light assembly 15 that cooperates with the camera assembly to provide visualization of a body cavity. The term “light assembly” broadly refers to any element that can be used to illuminate a camera's field of view. Thus, light assembly 15 can include one or more light emitting diodes (LEDs, semiconductor devices that emit light when current flows through them), fiber optic light sources, a white light LED, an infrared (750 nm to 1000 μm) light LED, a near infrared (870 to 1100 nm) light LED, an ultraviolet (100-400 nm) light LED, cold cathode fluorescent lamp (CCFLs, a type gas-discharge lamp similar to linear fluorescent or compact fluorescent lamps), diode laser (a semiconductor device similar to a light-emitting diode in which a diode pumped directly with electrical current can create lasing conditions at the diode's junction), diode pumped solid state laser, organic light-emitting diode (OLED, a type of light-emitting diode (LED) in which the emissive electroluminescent layer is an organic compound film that emits light in response to an electric current), or combinations thereof. The light assembly also comprises a light source drive circuit for driving the emission of light.

The camera and light assemblies are therefore used to assist a medical professional in visualizing a body cavity to detect abnormalities, such as colon cancer and polyps (a growth of tissue that includes a stalk that attaches the growth). The camera provides the medical professional with a two-dimensional and/or three-dimensional view from the distal end of the endoscope. The camera and lighting systems can be powered through associated wiring, as discussed in detail below. Alternatively, the camera and lighting systems can be battery powered or rechargeable.

The capsule further includes magnet 20 that can be used to propel the device through the body, to a desired location. The term “magnet” as used herein refers to a material that both produces its own magnetic field and responds to magnetic fields. A magnetic field is invisible but is responsible for pulling on other ferromagnetic materials (such as iron) and attracting or repelling other magnets. Magnet 20 can be a permanent magnet that remains magnetized. Specifically, permanent magnets are constructed from a material that is magnetized and creates its own persistent magnetic field. In other embodiments, magnet 20 can be an impermanent magnet that loses its memory of previous magnetizations.

Magnet 20 can be formed from any suitable material, such as (but not limited to) steel (e.g., a steel connector), neodymium, samarium cobalt, ceramic, alnico (aluminum nickel cobalt), iron, nickel, cobalt, some alloys of rare earth metals, and some naturally occurring minerals (such as lodestone).

Magnet 20 permits motion and orientation control of the capsule by externally generated magnetic fields. These fields can be generated by external magnet 30 that is present on the exterior of the patient's body and controlled by a health care professional. One embodiment of external magnet 30 is illustrated in FIGS. 3a and 3b. In some embodiments, external magnet 30 can be an electromagnet that can be magnetized by electric current, which can be modulated to adjust the strength of the magnet. Thus, the external magnet can be configured as an adjustable electromagnet in some embodiments. External magnet 30 can be constructed from any suitable magnetic or electromagnetic material, such as (but not limited to) neodymium, samarium cobalt, ceramic, and/or alnico (aluminum nickel cobalt). In some embodiments, magnets 20, 30 can have a strength of about 100-14,000 gauss (e.g., at least/no more than about 100, 200, 300, 400, 500, 600, 700, 800, 900,1000, 1500, 2000, 2500, 3000, 3500, 4000, 4500, 5000, 5500, 6000, 6500, 7000, 7500, 8000, 8500, 9000, 9500, 10000, 10500, 11000, 11500, 12000, 12500, 13000, 13500, 14000 gauss). However, it should be appreciated that the strength of the magnets is not limited to the ranges given above.

Magnets 20 and 30 can have any shape, such as cylindrical, conical, elliptical, or cubical shape.

As set forth above, capsule 5 includes covering 25 that surrounds the capsule internal components (e.g., camera assembly, light assembly, internal magnet). The covering can be constructed from any suitable material, such as (but not limited to) a polymeric material. It should be appreciated that the covering is constructed from a material that does not impede magnetic attraction between the internal and external magnets 20, 30. Suitable polymeric materials can include (but are not limited to) polyacrylates, polyethylenes, polyamides, polystyrenes, polyisoprenes, polycarbonates, polyesters, polyureas, polyurethanes, polyolefins, polysaccharides, epoxy resins, vinyl polymers, urea cross-linked with formaldehyde or gluteraldehyde, melamine cross-linked with formaldehyde, gelatin-polyphosphate coacervates optionally cross-linked with gluteraldehyde, gelatin-gum arabic coacervates, cross-linked silicone fluids, polyamines reacted with polyisocyanates, acrylate monomers polymerised via free radical polymerization, silk, wool, gelatine, cellulose, alginate, proteins, and combinations thereof.

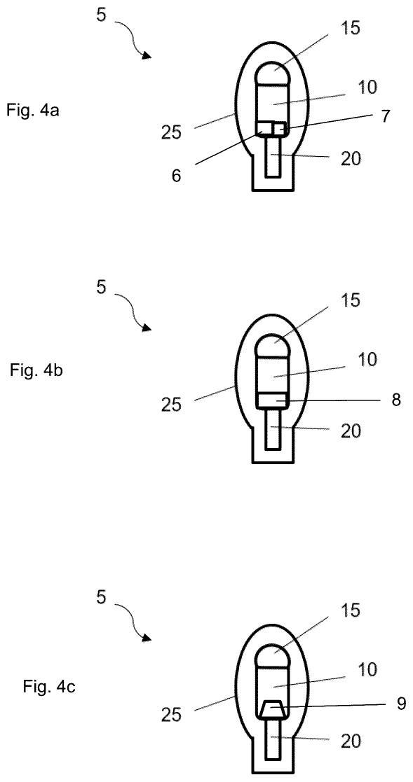

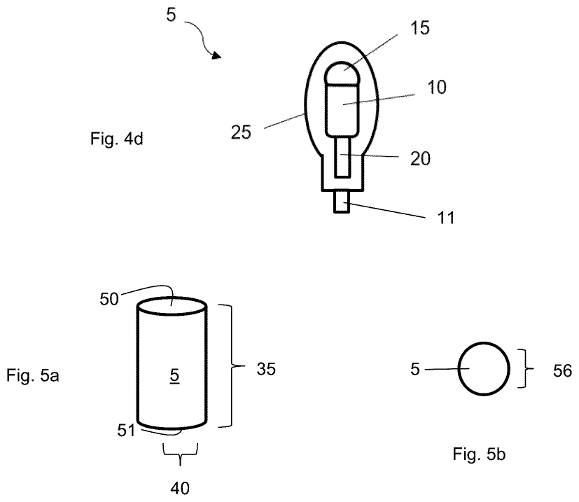

Optionally, capsule 5 can include a power source 6 and receiver 7 to transmit images taken by the camera assembly and communicate with a computer or other receiving device, as shown in FIG. 4s. The term “power source” refers an apparatus, a device, a system, and/or any combination thereof that generates, transmits, converts, and/or supplies power or energy, including, but not limited to, electrical power. The term “receiver” refers to a device that receives information and converts the information to usable form.

In some embodiments, the capsule can include a gyroscope 8 and/or accelerometer 9 for positional information, as illustrated in FIGS. 4b and 4c. The term “gyroscope” refers to a device that measures orientation and angular velocity of the capsule. The term “accelerometer” refers to a device that measures proper acceleration or speed of the capsule. The gyroscope and/or accelerometer provides the ability to map the positional progress of capsule 5 in real time. For example, a positional map of the entire exam can be created, areas of interest marked, and visual images generated corresponding to each positional location of the capsule. As another example, three gyroscopes can be used to provide orientation data about three different axes of rotation.

The capsule can also provide connection 11 that allows for cooperation with a water and/or air hose, allowing for suitable visibility within the internal cavity of the patient, as shown in FIG. 4d. For example, to give the light assembly and camera assembly proper space to work, air and/or water can be optionally pumped into the cavity.

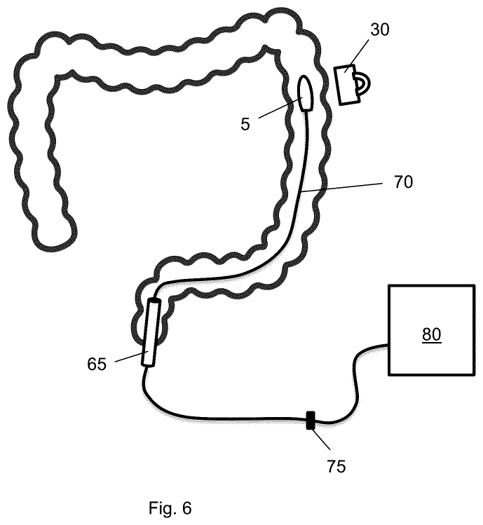

Capsule 5 can be constructed in any suitable size. For example, the capsule can have length 35 of about 2 cm or less (e.g., at least/no more than about 2, 1.5, or 1 cm), as illustrated in FIG. 5a. The term “length” refers to the longest horizontal distance of the capsule from first end 50 to opposed second end 51. The capsule can further include a diameter 56 of about 1 cm or less, as shown in FIG. 5b. However, it should be appreciated that the capsule can be configured in any desired length and diameter and is not limited to the ranges given herein. It should further be appreciated that the capsule dimension can vary depending on patient size (e.g., child versus adult) and on the body region being examined by the medical practitioner.

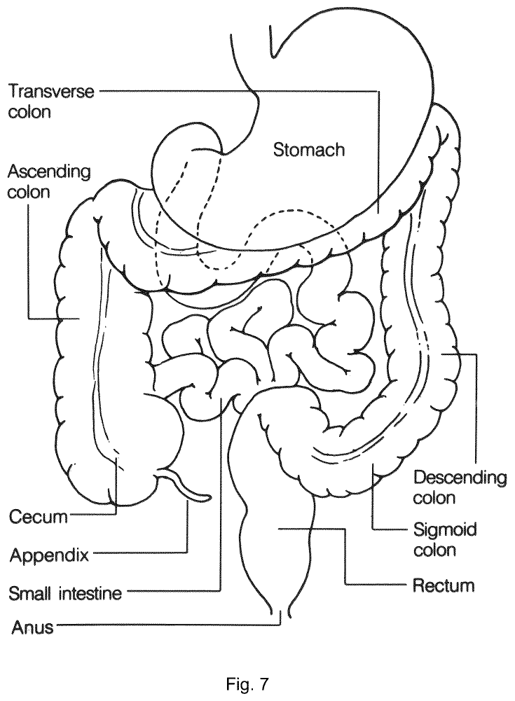

Capsule 5 can be used to perform colonoscopy imaging as set forth above. As shown in FIG. 6, the capsule can be inserted through the patient's rectum. Prior to insertion, the patient is required to empty the colon to allow for clear imaging of the patient tissues. In some embodiments, tubing 65 can be used to facilitate insertion of the capsule into the rectum. The tubing also functions to prevent wire 70 from damaging the rectum of the patient. The capsule can then be maneuvered through the lower GI tract (e.g., rectum, colon, large and small intestines) using external magnet 30. The rectum, colon, large intestine, and small intestine are illustrated in FIG. 7. For example, the external magnet can be placed on an external surface of the patient's body or skin. Magnetic attraction between the external magnet and the internal magnet of capsule 5 allows the capsule to travel the same path as the external magnet. The magnetic force can be applied at an angle of about 15-80 degrees (e.g., at least/no more than about 15, 20, 25, 30, 35, 40, 45, 50, 55, 60, 65, 70, 75, or 80 degrees). Notably, the patient bed is required to be constructed without materials that interfere with the magnetic field to avoid interfering with the disclosed method.

In this way, the path of the capsule (and associated camera and light assembly) can be easily directed by the medical professional. As the capsule travels through the body, the light assembly and camera assembly cooperate to capture images (e.g., photos or a live feed video) that are transmitted via wire link 70 in communication with connector 75 of a computer or other image receiving device 80. To precisely locate the position of observed abnormal growth in the colon, scales in centimeters and inches can be marked on the wire. The turning points inside the colon can be coordinated by moving the magnetic force according to the image feedback from the camera within the capsule to a computer monitor. As such, the medical professional can view the patient's lower GI tract on demand as the capsule passes through the cavity. If an area of concern appears or the professional wishes to get a better or different view, capsule 5 can be reversed using a magnetic field provided by external magnet 30. Alternatively or in addition, the capsule can be rotated to give a different view of a particular area. After the viewing, the capsule can be maneuvered out of the body through the rectum.

While the disclosed system has been discussed primarily in reference to colonoscopy procedures, it can be used in a variety of medical procedures in which imaging of a body tissue, organ, cavity, or lumen is required. For example, capsule 5 can be useful in anoscopy, bronchoscopy, colonoscopy, cystoscopy, esophagogastroduodenoscopy (EGD), laparoscopy, and sigmoidoscopy procedures.

Further, the disclosed system and method has been discussed primarily in reference to human patients. However, the term “patient” is broadly construed and can include veterinary subjects (e.g., cats, dogs, horses, bird, sheep, cows, bulls, goats, and the like).

The disclosed magnetically driven capsule system offers many advantages over prior art visualization methods. For example, capsule 5 allows the medical professional to precisely position a camera to focus on a particular area of concern through Al.

The disclosed system allows for repeated passing of the capsule in a desired area (e.g., capsule 5 can be moved forward, backward, left, right, rotated), allowing for improved detection of abnormalities that would otherwise be missed.

Advantageously, capsule 5 does not require a battery or other power source. Conventional capsules have a battery life of about 8 hours, which can be problematic if transit to a specific area is slow (e.g., allowing only a portion of the small intestine and/or large intestine to be examined before the battery fails).

The disclosed system is more acceptable to patients, requiring no sedation and minimal or no discomfort.

Safety concerns associated with conventional colonoscopy procedures are also minimized, such as tearing of the intestines, discomfort, bleeding, and the like.

Conventional colonoscopy methods require extensive training for staff in the efficient handling and maintenance of the scope systems and equipment. In comparison, the disclosed system is easy to use and requires minimal training by staff to successfully perform a procedure.

Maintaining conventional equipment used during colonoscopy procedures is of vital importance as improperly reprocessed endoscopes can lead to cross-contamination and can potentially expose patients to infections. Because no colonoscopy or endoscope is required in the disclosed system or method, such concerns are eliminated.

The foregoing description has been set forth merely to illustrate the invention and are not intended to be limiting. Each of the disclosed aspects and embodiments of the invention may be considered individually or in combination with other aspects, embodiments, and variations of the invention. In addition, unless otherwise specified, the steps of the methods of the invention are not confined to any particular order of performance. Modifications of the disclosed embodiments incorporating the spirit and substance of the invention may occur to persons skilled in the art, and such modifications are within the scope of the invention.

What is claimed is:

1. A magnetically driven capsule comprising:
a camera assembly configured to take images, provide video, or both;
a light assembly configured to illuminate areas surrounding the camera assembly;
an internal magnet;
an outer covering surrounding the camera assembly, light assembly, and magnet.

2. The magnetically driven capsule of claim 1, wherein the camera assembly comprises a digital camera or a digital video camera.

3. The magnetically driven capsule of claim 1, wherein the camera assembly is configured to provide a two-dimensional or three-dimensional image.

4. The magnetically driven capsule of claim 1, wherein the light assembly comprises one or more light emitting diodes (LED).

5. The magnetically driven capsule of claim 1, wherein the internal magnet is a permanent magnet that remains magnetized.

6. The magnetically driven capsule of claim 1, wherein the internal magnet comprises steel, neodymium, samarium cobalt, ceramic, aluminum nickel cobalt, iron, nickel, cobalt, alloy of rare earth metals, lodestone, or combinations thereof.

7. The magnetically driven capsule of claim 1, further comprising a power source and communication source configured as a wire attached to an external supply.

8. The magnetically driven capsule of claim 1, further comprising an accelerometer, a gyroscope, or both.

9. The magnetically driven capsule of claim 1, comprising a length of about 2 centimeters or less and a diameter of about 1 centimeter or less.

10. A system for guiding a magnetically driven capsule, the system comprising:
a magnetically driven capsule defined by:
a camera assembly configured to take images, provide video, or both;
a light assembly configured to illuminate areas surrounding the camera assembly;
an internal magnet;
an outer covering surrounding the camera assembly, light assembly, and magnet;
an external magnet that exhibits magnetic attraction to the magnet sufficient to guide a location of the magnetically driven capsule.

11. The system of claim 10, wherein the external magnet is an electromagnet.

12. The system of claim 10, wherein the external magnet comprises neodymium, samarium cobalt, ceramic, aluminum nickel cobalt, or combinations thereof.

13. The system of claim 10, wherein the internal magnet is a permanent magnet that remains magnetized.

14. The system of claim 10, wherein the internal magnet comprises steel, neodymium, samarium cobalt, ceramic, aluminum nickel cobalt, iron, nickel, cobalt, alloy of rare earth metals, lodestone, or combinations thereof.

15. A method of visualizing a body cavity, the method comprising:
positioning a magnetically driven capsule within a body cavity to be visualized, the magnetically driven capsule defined by:
a camera assembly configured to take images, provide video, or both;
a light assembly configured to illuminate areas surrounding the camera assembly;
an internal magnet;
an outer covering surrounding the camera assembly, light assembly, and magnet;
maneuvering the magnetically driven capsule along a desired path using an external magnet positioned on an external surface of the patient's body, whereby magnetic attraction between the internal and external magnets guide the path of the capsule;
whereby the body cavity is visualized.

16. The method of claim 15, wherein the body cavity is selected from the rectum, large intestine, small intestine, or combinations thereof.

17. The method of claim 15, wherein the magnetically driven capsule can be maneuvered via the external magnet in a forward direction, a backward direction, to the right, to the left, rotate, or combinations thereof.

18. The method of claim 15, wherein the positioning is accomplished by a plastic tubing sized to allow the magnetically driven capsule to pass therethrough.

19. The method of claim 15, wherein the magnetic force of the external magnet is applied to the internal magnet at an angle of about 15-80 degrees.

20. The method of claim 15, wherein the magnetic force of the external magnet is applied to the internal magnet at an angle of about 30-60 degrees.
